(19) United States
(12) Patent Application Publication
(10) Pub. No.: US 2005/0124993 A1
(43) Pub. Date: Jun. 9, 2005

(54) Facet fusion system

(76) Inventor: James L. Chappuis, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/037,994

(22) Filed: Jan. 18, 2005

Related U.S. Application Data
(63) Continuation-in-part of application No. 10/725,832, filed on Dec. 2, 2003.
(60) Provisional application No. 60/430,311, filed on Dec. 2, 2002.

(51) Int. Cl. A61F 2/30; A61B 17/58; A61F 5/00

(52) U.S. Cl. 606/61

(57) ABSTRACT

Included in the present disclosure is a system and method for fusing a facet joint. The system includes a trochar arranged and configured for use during percutaneous retraction, a retractor arranged and configured for use during percutaneous retraction, and a facet bur arranged and configured for decorticating the facet joint. Also included is at least one implant configured for insertion into the facet joint. The system also implements the trochar, retractor, and facet bur to prepare the facet joint for fusion.

### CROSS-REFERENCE TO RELATED APPLICATION

This application is a continuation-in-part of co-pending U.S. utility patent application entitled, “Facet Fusion System,” having Ser. No. 10/725,832, filed on Dec. 2, 2003, which is entirely incorporated herein by reference. Co-pending U.S. utility patent application entitled “Facet Fusion System,” having Ser. No. 10/725,832 claims priority to co-pending U.S. provisional application entitled, “Facet Fusion Apparatus and Method of Use,” having Ser. No. 60/430,311, filed on Dec. 2, 2002, which is entirely incorporated herein by reference.

### TECHNICAL FIELD

The present disclosure generally relates to surgical instruments, and in particular, relates to a facet fusion system.

### BACKGROUND

Skeletal structures are formed of bones and adjoining structures which include cartilage, for instance. For various reasons, these skeletal structures may require artificial support or stabilization. For example, the human spine is composed of a column of thirty-three bones, called vertebrae, and their adjoining structures. The twenty-four vertebrae nearest the head are separate bones capable of individual movement and generally are connected by anterior and posterior longitudinal ligaments and by discs of fibrocartilage, called intervertbral discs, positioned between opposing faces of adjacent vertebrae. Each of these vertebrae includes a vertebral body and a dorsal arch that enclose an opening, called the vertebral foramen, through which the spinal cord and spinal nerves pass. The remaining nine vertebrae are naturally fused to form the sacrum and the coccyx and are incapable of individual movement.

Each vertebra capable of individual movement is joined to the adjoining vertebra at facet joints. Facet joints allow for movement of the spine in all directions. Arthritis, degenerative disc disease, and other various degenerative conditions can result in the need to surgically fuse the facet joints together.

Facet joint fusion can reduce or eliminate pain and/or complications experienced by patients with degenerating facet joints. Currently, facet joints are fused by decorticating the joint in an open procedure followed by inserting a bone implant. In this process, often times the facet joint is not completely decorticated resulting in a low fusion success rate.

Thus, a heretofore unaddressed need exists in the industry to address the aforementioned deficiencies and inadequacies.

### SUMMARY

Preferred embodiments of the present disclosure provide a facet fusion system for fusing a facet joint. Briefly described in architecture, one embodiment of the system can be implemented as follows. A facet fusion system comprises a trochar and a retractor, both being arranged and configured for use during percutaneous retraction. A facet bur is adapted for decorticating the facet joint. The trochar, retractor, and facet bur are implemented to prepare the facet joint for fusion.

Other systems, methods, features and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

The embodiments of present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a diagram illustrating two vertebrae within the human spine.

FIG. 2 is a functional block diagram of one embodiment of a trochar used in facet fusion surgery on the facet joint of FIG. 1.

FIG. 3 is a perspective view diagram of a retractor used in facet fusion surgery on the facet joint of FIG. 1.

FIG. 4 is a perspective view diagram of a facet bur used in facet fusion surgery on the facet joint of FIG. 1.

FIG. 5 is a perspective view diagram of a sizing insert used in facet fusion surgery on the facet joint of FIG. 1.

FIG. 6 a perspective view diagram of a trapezoidal implant used in facet fusion surgery on the facet joint of FIG. 1.

FIG. 7 is a perspective view diagram of a T-shaped implant used in facet fusion surgery on the facet joint of FIG. 1.

FIG. 8 is a perspective view diagram of the trapezoidal implant of FIG. 6, further including a cage construction.

FIG. 9 is a perspective view diagram of the T-shaped implant of FIG. 7, further including a cage construction.

FIG. 10 is a functional block diagram of a facet joint with the trapezoidal cage implant of FIG. 8 and stapling device.

FIG. 11 is a functional block diagram of a facet joint with the T-shaped cage implant of FIG. 9 and stapling device.

### DESCRIPTION OF THE PREFERRED EMBODIMENTS

Having summarized various aspects of the present disclosure, reference will now be made in detail to the description as illustrated in the drawings. While the disclosure will be described in connection with these drawings, there is no intent to limit it to the embodiment or embodiments disclosed therein. On the contrary, the intent is to cover all alternatives, modifications, and equivalents included within the spirit and scope of the invention as defined by the appended claims. It should be emphasized that many variations and modifications may be made to the above-described embodiments. All such modifications and variations are intended to be included herein within the scope of this disclosure and protected by the following claims.

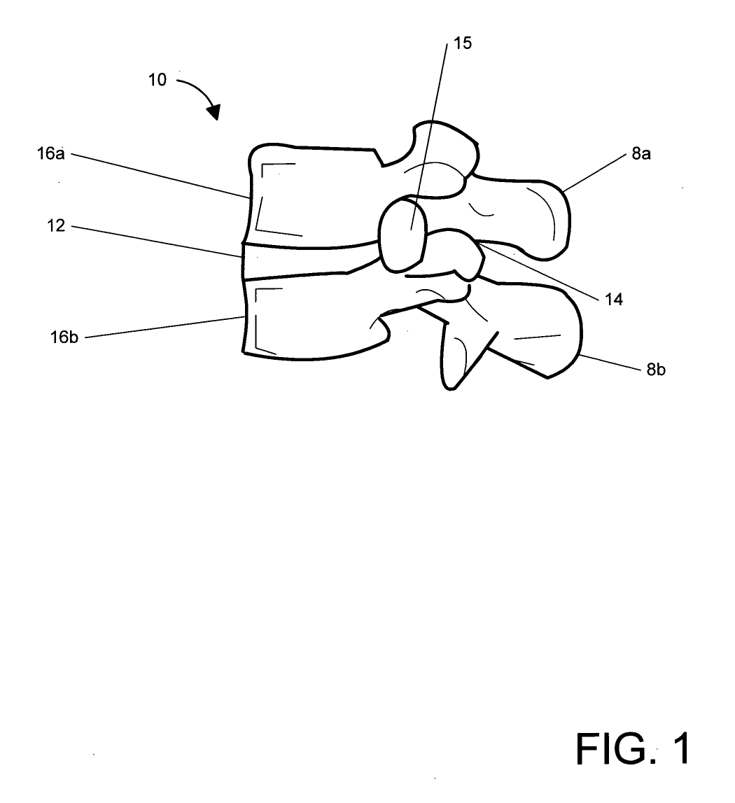

FIG. 1 is a diagram illustrating two vertebrae within the human spine. As shown in FIG. 1, spinal column 10 includes two vertebrae 16a and 16b. Intervertebral disk 12 is located between vertebrae 16a and vertebrae 16b. Invetertebral disk 12 serves to cushion the vertebrae from impact due to bodily movement.

As stated above, each of the vertebrae includes a vertebral body and a dorsal arch that enclose an opening, called the vertebral foramen 15, through which the spinal cord and spinal nerves pass. The vertebral foramen is the opening between every two vertebrae where the nerve roots exit the spine. The nerve roots travel through the foramen to reach the rest of the body. Without the foramen, nerve signals could not travel to and from the brain to the rest of the body.

Also included in FIG. 1 are facets 8a and 8b and facet joint 14. Facets 8a and 8b are the “bony knobs” that meet between each vertebra to form the facet joints 14 that join your vertebrae together. There are two facet joints between each pair of vertebra, one on each side. They extend and overlap each other to form a joint between the neighboring vertebra facet joints.

The facet joints 14 are what are known as synovial joints. A synovial joint, such as the knee or elbow, is a structure that allows movement between two bones. In a synovial joint, the ends of the bones are covered with a material called articular cartilage. This material is a slick spongy material that allows the bones to glide against one another without much friction.

Surrounding facet joint 14 is a watertight sack made of soft tissue and ligaments. This sack creates what is called the “joint capsule.” The ligaments are soft tissue structures that hold the two sides of the facet joint together. The ligaments around the facet joint combine with the synovium to form the joint capsule that is filled with fluid (synovial fluid). This fluid lubricates the joint to decrease the friction, just like oil lubricates the moving parts of a machine.

As stated above, fusion of a facet joint 14 may be required when a patient suffers from arthritis, or other degenerative disease which makes movement painful in the spinal region.

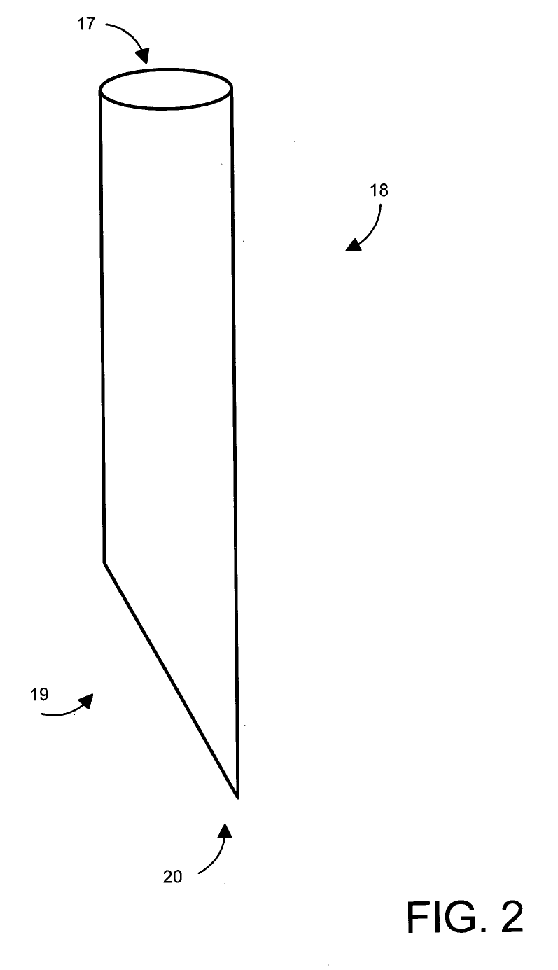

FIG. 2 is a perspective view diagram of one embodiment of a trochar 18 used in facet fusion surgery on the facet joint 14 of FIG. 1. As shown in FIG. 2, the trochar 18 comprises a hollow body portion 17 having at least one tapered end 19. The trochar 18 preferably includes a substantially sharpened, pointed tip 20 toward the tapered end 19. This device may be used for decorticating the facet joint 14 in order to prepare for the facet fusion surgery.

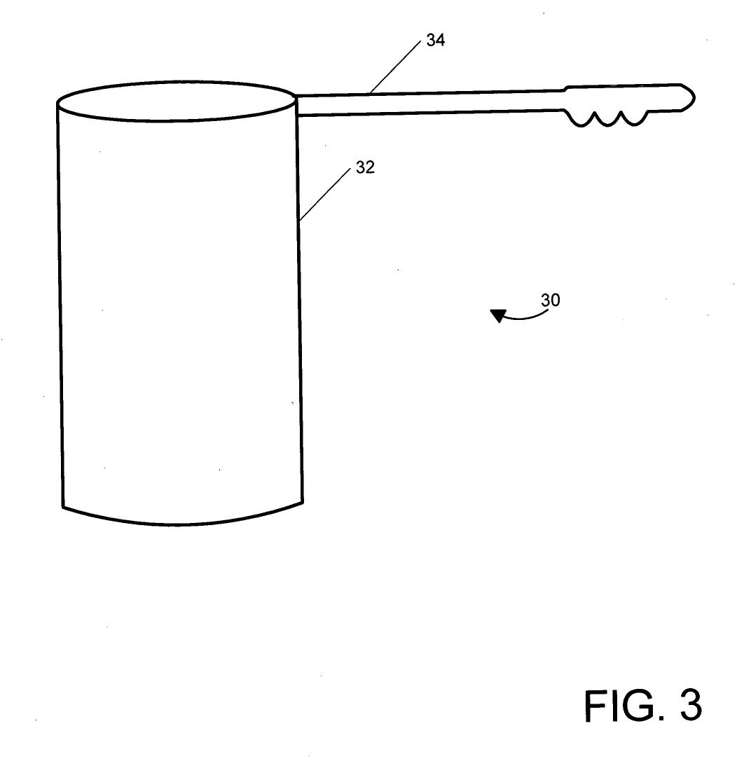

FIG. 3 is a perspective view diagram of a retractor 20 used in facet fusion surgery on the facet joint 14 of FIG. 1. As shown in FIG. 3, the retractor 20 comprises a main body portion 32 having a handle 34 extending therefrom. The main body portion 32 is preferably hollow. The trochar 18 and the retractor 20 are used to perform percutaneous retraction and dissection of the facet joint 14 in a manner known to those skilled in the art. More specifically, the trochar 18 is placed into a position such as to dilate surrounding muscle. The retractor 30 is then placed over the facet joint 14. The retraction and dissection partially prepares the facet joint 14 for fusion.

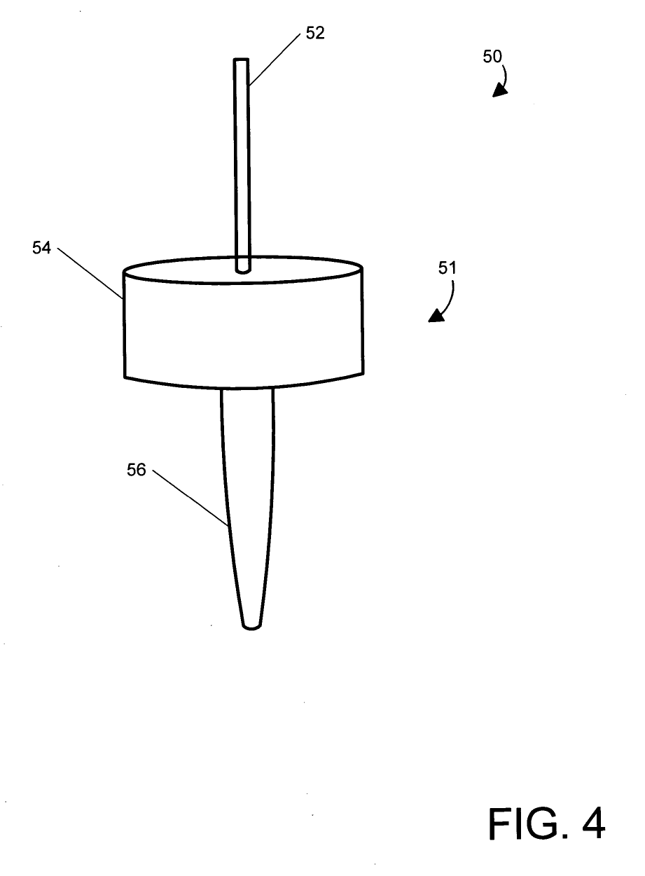

FIG. 4 is a perspective view diagram of a facet bur 50 used in facet fusion surgery on the facet joint 14 of FIG. 1. As shown in FIG. 4, the facet bur 50 comprises a head 51 having a substantially T-shaped cross-section. The head 51 comprises an extension 56 and a planar surface 54. The facet bur 50 includes a shaft 52 extending from the head 51. The shaft 52 is adapted to releasably engage a power source for rotation, such as a surgical drill, or the like. The head 51 is arranged and configured to decorticate the facet joint 14 upon being engaged therewith while rotating at a desired speed, thereby tapering the facet joint 14 into a substantially wedge shaped configuration. While tapering the facet joint 14 with the extension 56, the planar surface 54 disposed substantially adjacent the extension 56 engages a posterior surface of the facets 8a or 8b in order to plane the surface thereof. The extent to which the top of each facet 8a or 8b is planed is determined by the configuration of the implant to be used. Depending on the size and shape of the implant used, a greater or lesser degree of planing is required.

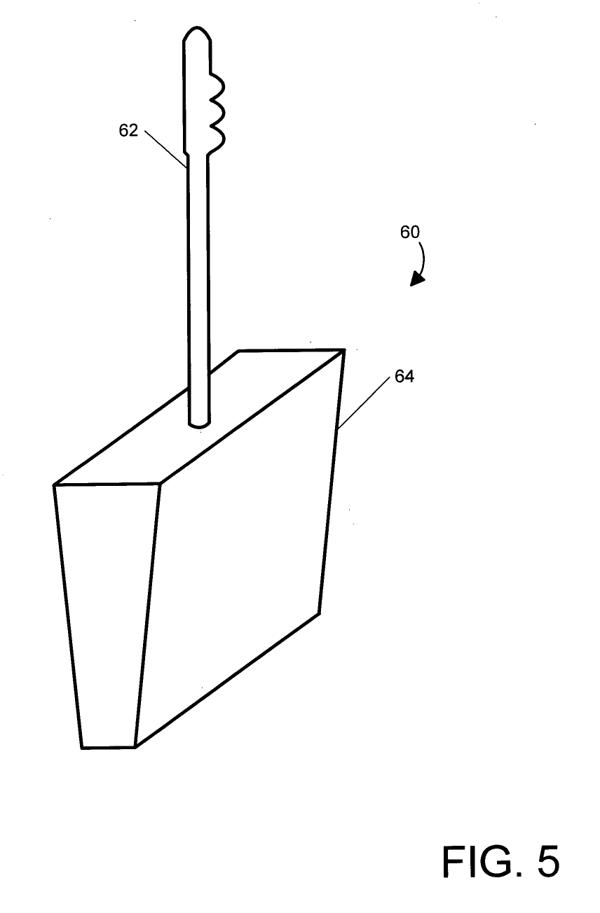

FIG. 5 is a perspective view diagram of a sizing insert 60 used in facet fusion surgery on the facet joint 14 of FIG. 1. As shown in FIG. 5, the sizing insert 60 comprises a body portion 64 and a handle 62. After the facet to be fused has been located, decorticated, and planed as desired and described herein, the sizing insert 60 is inserted into the area of the facet joint 14 where an implant will be positioned for fusion. As is obvious to one of ordinary skill in the art, the system of the present disclosure preferably comprises a plurality of sizing inserts 60, each having at least a slight dimension variation. If a particular sizing insert 60 does not fit, a sizing insert 60 of a different size will be placed into the facet joint 14 until the proper size is determined. The handle 62 allows for easy insertion and removal of the sizing insert 60.

It should be understood that although the body portion 64 is illustrated as being substantially wedge-shaped, it may comprise any suitable configuration. Sizing inserts 60 of various dimensions can be placed in the facet one after the other until the user can ascertain the necessary implant size to be placed in the facet cavity for fusion.

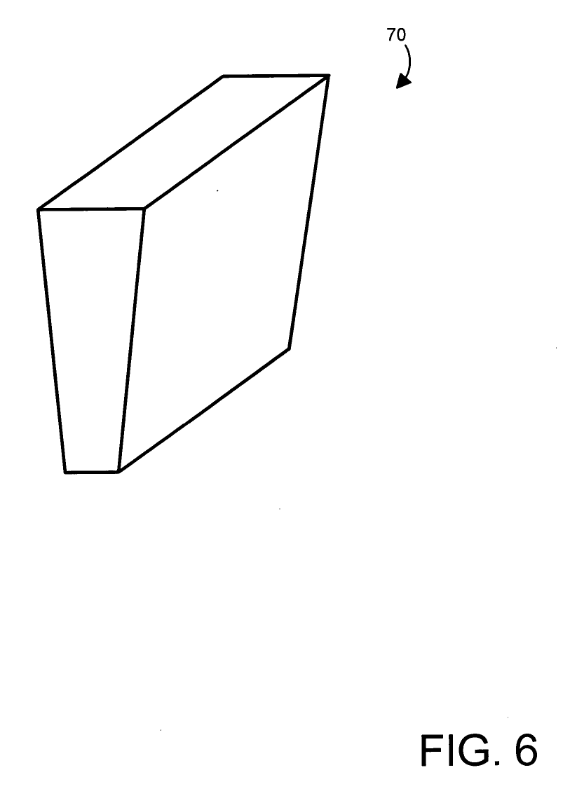

FIG. 6 a perspective view diagram of a trapezoidal implant 70 used in facet fusion surgery on facet the joint 14 of FIG. 1. As shown in FIG. 6, the trapezoidal implant 70 is positioned in the facet joint cavity 14 for fusion. The trapezoidal implant 70 should preferably correspond in size and shape to the dimension to the selected sizing insert 60, as stated above. The burring of the facets 8a and 8b and wedging of the trapezoidal implant 70 into position results in a sufficient amount of friction to hold trapezoidal implant 70 in the desired position. The trapezoidal implant 70 may comprise bone, coral, or any suitable material lending itself to fusion in such an environment.

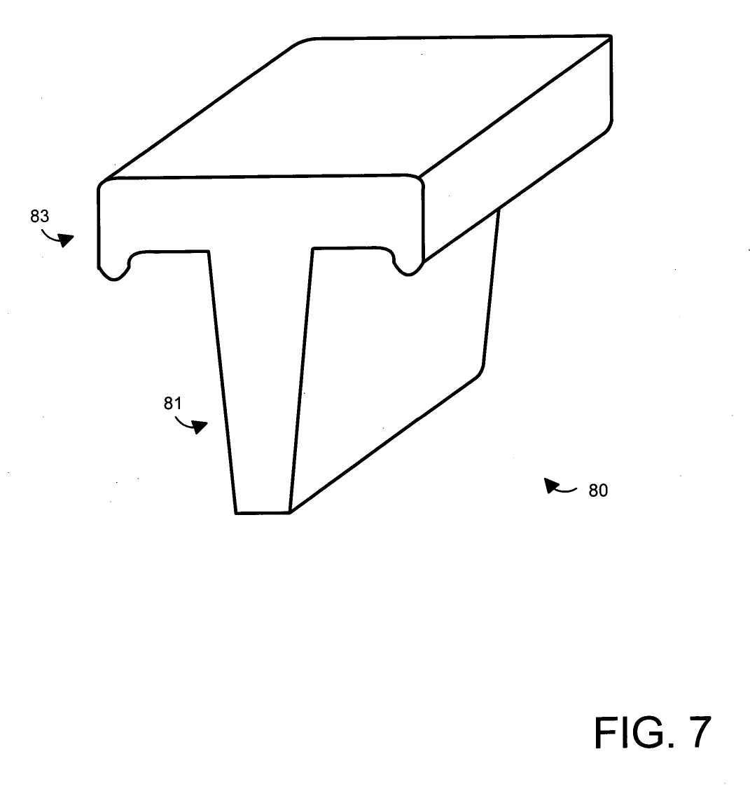

FIG. 7 is a perspective view diagram of a T-shaped implant 80 used in facet fusion surgery on the facet joint 14 of FIG. 1. As shown in FIG. 7, the embodiment of the T-shaped implant 80 is synthetic. The synthetic T-shaped implant 80 comprises a fusion portion 81 and a cap 83. The fusion portion 81 is adapted to engage an internal portion of the facet joint 14. The cap 83 is adapted to engage a posterior portion of the facet joint 14 in order to secure the T-shaped implant 80 in the desired position. The fusion portion 81 is illustrated as having a tapered cross-section, however, it should be understood that the fusion portion 81 may comprise any suitable configuration. The synthetic T-shaped implant 80 may comprise polished stainless steel, high-density polyethylene, or any suitable material. Similar to the trapezoidal implant 70, the T-shaped implant 80 is selected in a size and configuration substantially corresponding to that indicated as appropriate by the sizing insert 60. The burring of the facet and wedging of an appropriately sized and configured implant into position results in a sufficient amount of friction to hold the implant 80 in the desired position.

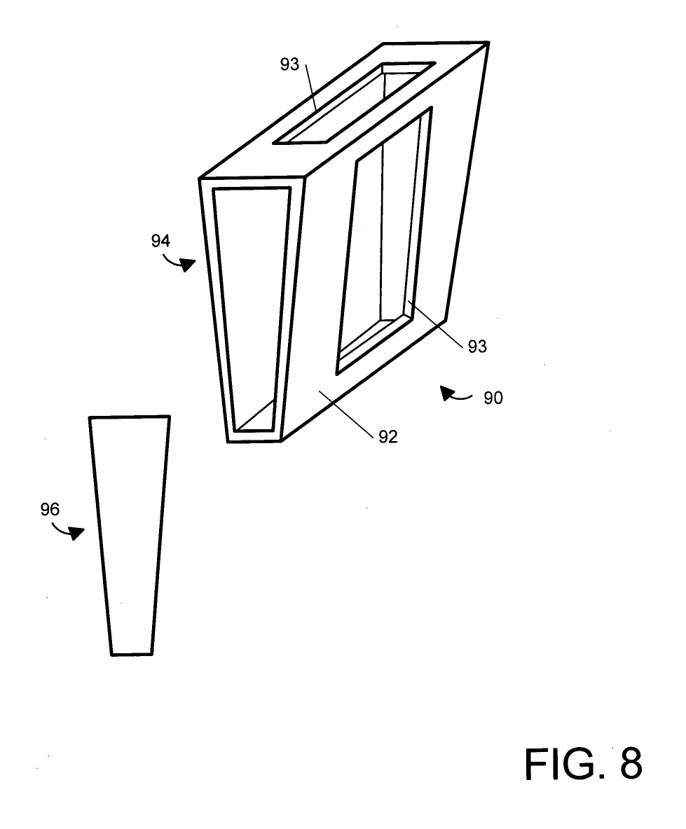

FIG. 8 is a perspective view diagram of the trapezoidal implant 70 of FIG. 6, having a cage construction. As shown in FIG. 8, the implant 90 is configured with fusion apertures 93 on various portions of the implant 90 to form a cage-like member 92. The fusion apertures 93 allow the bone access to an inner cavity 94 defining a volumetric area into which graft material can be placed. As is evident, the fusion apertures 93 may be any size shape or number, and may be located anywhere on implant 90, depending on the particular situation.

In addition, a lid 96 can also be included and can be removed from the end of the implant 90, thereby opening the cavity 94 to provide access thereto. Fusion material such as bone morphogenic protein (BMP) or polyether ether keyton (PEEK) may be inserted into the cavity 94 of the implant 90. A collagen-based sponge, or other similar material, may be used as a carrier material for the BMP solution. The BMP infused sponge may be inserted into trapezoidal implant 90. As the trapezoidal implant 90 is subjected to pressure once it is positioned in facet joint 14, the fusion material fuses with the facet joint 14 to solidify the fusion. The fusion apertures 93 allow for fusion of the surrounding bone to fusion material disposed within the implant 90.

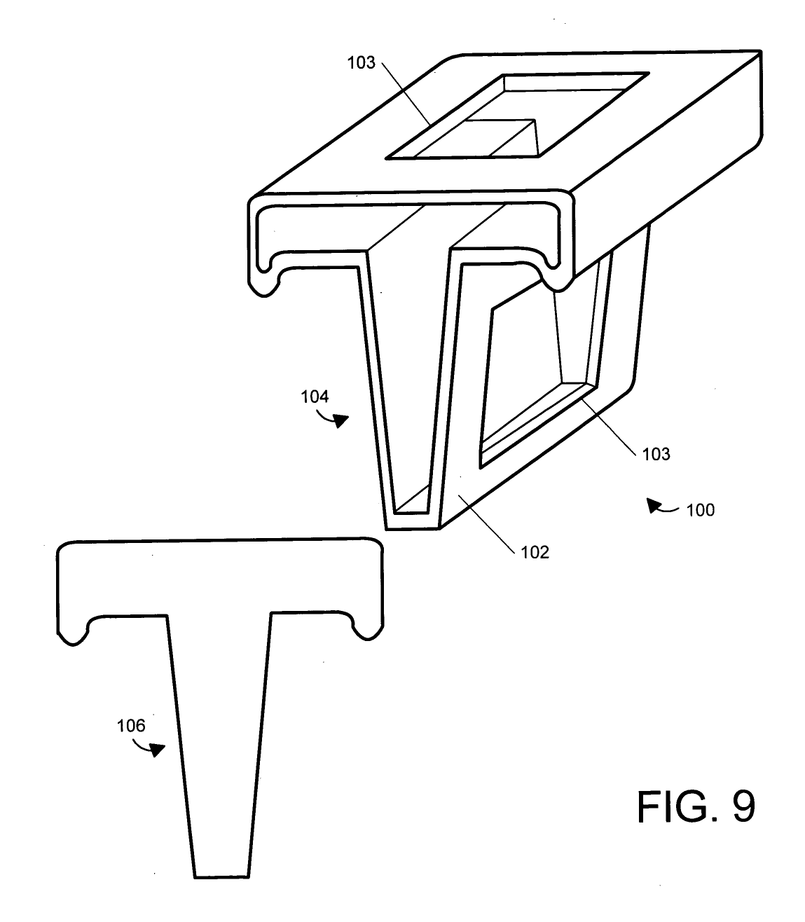

FIG. 9 is a perspective view diagram of the T-shaped implant 80 of FIG. 7, further including a cage construction. As shown in FIG. 9, a synthetic T-shaped implant 100 can also be implemented with fusion apertures 103 to form a cage-like member. As in FIG. 8, the fusion apertures 103 may be any size, shape and number, and may be located on any portion of synthetic T-shaped implant 100.

The lid 106 may be removed from the end of T-shaped implant 100. Fusion material such as bone morphogenic protein (BMP) or polyether ether keyton (PEEK) may be inserted into the cavity 104 of the implant 90. A collagen-based sponge, or other similar material, may be used as a carrier material for the BMP solution. The BMP infused sponge may be inserted into the T-shaped implant 100. As the T-shaped implant 100 is subjected to pressure once it is positioned in the facet joint 14, the fusion material fuses with facet joint to solidify the fusion.

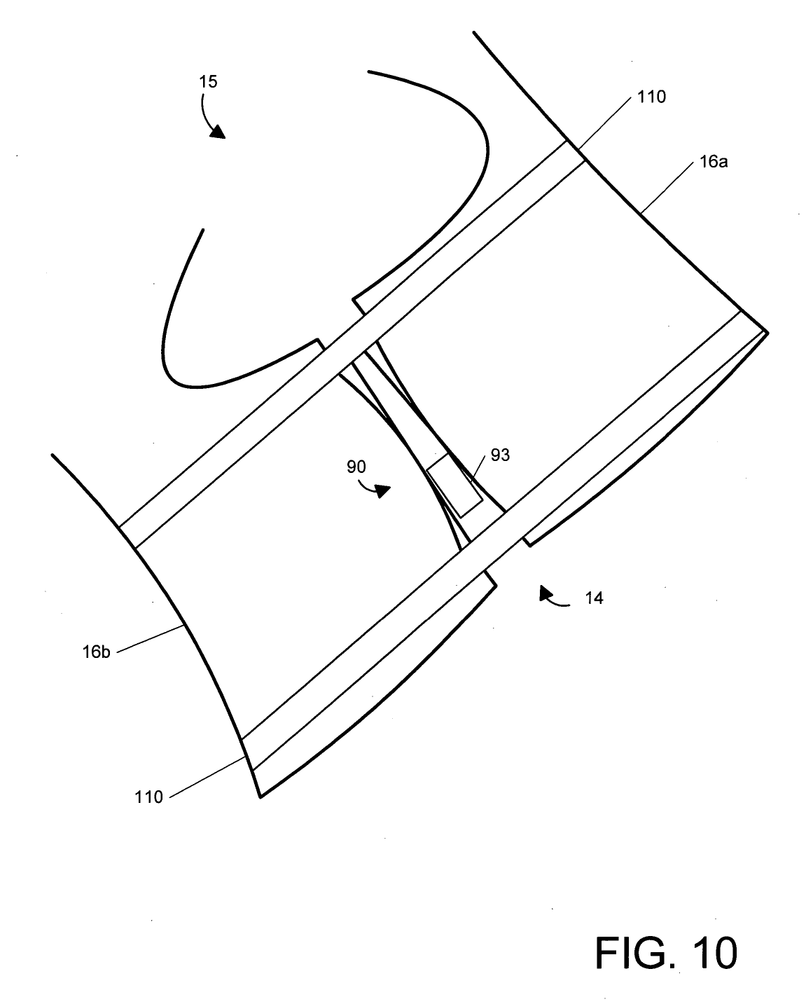

FIG. 10 is a functional block diagram of the facet joint 14 with the trapezoidal cage implant 90 of FIG. 8 and a stapling device 1 10. As shown in FIG. 10, the optional stapling device 110 may be implemented after the appropriate size trapezoidal cage implant 90 is selected and positioned in the facet joint cavity 14. This allows the optional stapling device 110 to engage the facet joint 14 and the trapezoidal cage implant 90, which included fusion aperture 93. The optional stapling device 110 secures the trapezoidal cage implant 90 in position in the facet joint 100. The optional stapling device 110 comprises any suitable material that can be heated to allow for some compression, such as Nitinol, or the like. As is evident from FIG. 10, the fusion aperture 93 allows an avenue for the bone to access the fusion material located within implant 90. This avenue facilitates bone growth, which allows for a successful facet fusion surgery.

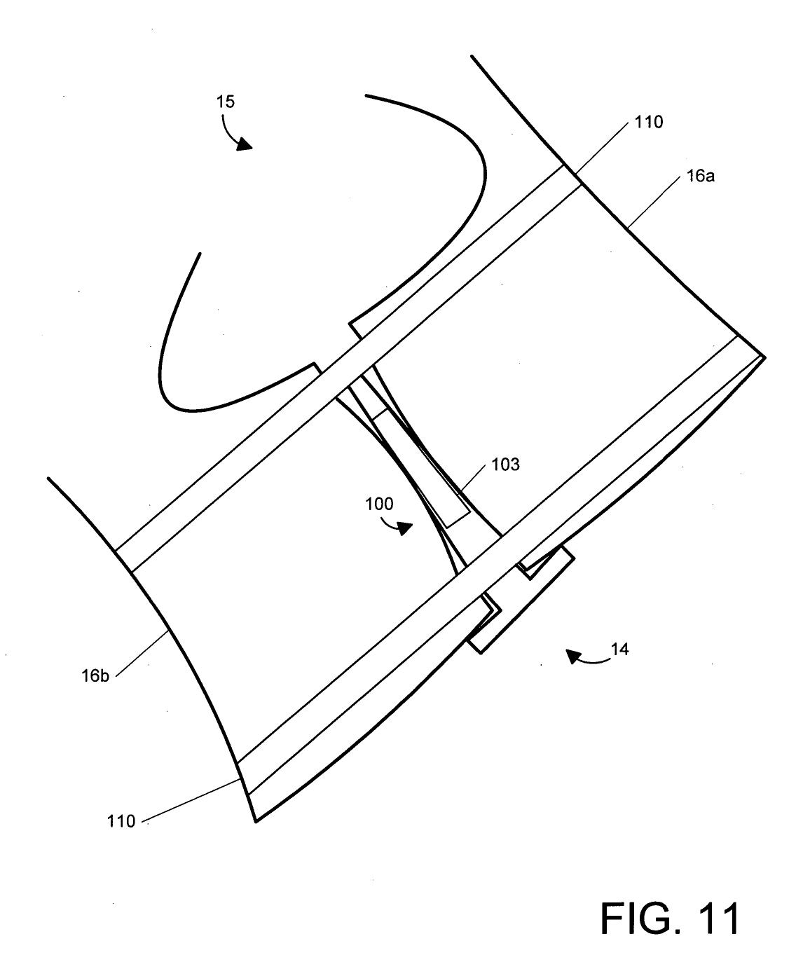

FIG. 11 is a functional block diagram of the facet joint 14 with the T-shaped cage implant 100 of FIG. 9 and a stapling device. As shown in FIG. 11, the optional stapling device 110 may be implemented after the appropriate size T-shaped cage implant 100 is selected and positioned in the facet joint cavity 14. This configuration allows the optional stapling device 110 to engage the facet joint 14 and the T-shaped cage implant 100. The optional stapling device 110 secures T-shaped cage implant 100 in position in the facet joint 100. The optional stapling device 110 comprises any suitable material that can be heated to allow for some compression, such as Nitinol, or the like. As is evident from FIG. 11, the fusion aperture 103 allows an avenue for the bone to access the fusion material located within implant 100. This avenue facilitates bone growth, which allows for a successful facet fusion surgery.

It should be emphasized that the above-described embodiments of the present disclosure, particularly, a “preferred” embodiment, are merely possible examples of implementations, merely set forth for a clear understanding of the principles of the disclosure. Many variations and modification may be made to the above-described embodiment(s) of the disclosure without departing substantially from the spirit and principles of the disclosure. All such modifications and variations are intended to be included herein within the scope of this disclosure and the present disclosure and protected by the following claims.

1. A system for fusing a facet joint comprising:
a trochar arranged and configured for use during percutaneous retraction;
a retractor arranged and configured for use during percutaneous retraction;
a facet bur arranged and configured for decorticating the facet joint; and
at least one implant configured for insertion into the facet joint;
wherein said trochar, said retractor, and said facet bur are implemented to prepare the facet joint for fusion.

2. The system of claim 1, wherein said facet bur further comprises:
a head having a planar surface and an extension extending from said planar; and
a shaft extending from said planar surface of said head, said shaft extending from said planar surface in a direction opposing said extension of said head, said shaft being arranged and configured to engage a source of rotation power for said rotation portion;
wherein said extension is arranged and configured to engage a facet joint to taper said facet joint and said planar surface is arranged and configured to engage and plane a posterior surface of the facet.

3. The system of claim 1, further comprising a sizing insert configured to aid in the determination of an appropriate size implant to be inserted into the facet joint to facilitate fusion.

4. The system of claim 3, wherein said sizing insert comprises:
a body portion; and
a handle extending from said body portion;
wherein said body portion is arranged and configured to aid in the determination of an appropriate size implant to be inserted into the facet joint to facilitate fusion.

5. The system of claim 1, wherein said implant comprises a cage configuration that comprises at least one fusion aperture.

6. The system of claim 5, wherein a fusion material is inserted into said implant.

7. The system of claim 6, wherein the fusion material comprises at least one of bone morphogenic protein and polyether ether keyton.

8. The system of clam 6, wherein a collagen based sponge is used as a carrier material for the fusion material.

9. The system of claim 1, wherein said implant is tapered for facilitating insertion into the facet joint.

10. The system of claim 1, wherein said implant comprises two sections, wherein the first section is positioned orthogonal to the second section, thereby forming a T-shape.

11. The system of claim 1, further comprising:
bonding means for fixing said implant in position in the facet joint;
wherein said bonding means facilitates fusion of the facet joint with said implant.

12. A system for fusing a facet joint comprising:
retraction means for performing percutaneous retraction; and
decorticating means for decorticating the facet joint.
planing means for planing a portion of the facet joint; and
insertion means for inserting an implant.

13. The system of claim 12, further comprising fusion means for facilitating fusion of the facet joint.

14. A method for fusing a facet joint, comprising:
performing percuntaneous retraction;
decorticating the facet joint; and
inserting an implant into the facet joint.

15. The method of claim 14, wherein said implant comprises a cage configuration, wherein the cage configuration comprises at least one fusion aperture.

16. The method of claim 15, further comprising inserting a fusion material into the implant.

17. The method of claim 16, wherein the fusion material comprises at least one of bone morphogenic protein and polyether ether keyton.

18. The method of claim 16, further comprising a collagen sponge configured for acting as a carrier material for the fusion material.

19. The method of claim 14, further comprising bonding the facet joint for facilitating fusion.
